(19) United States
(12) Patent Application Publication
(10) Pub. No.: US 2010/0015715 A1
(43) Pub. Date: Jan. 21, 2010

(54) MICROFLUIDIC DEVICE INCLUDING STRUCTURE THAT INCLUDES AIR VENT AND VALVE, AND METHOD OF TRANSFERRING FLUID USING THE SAME

(75) Inventors: Yoonkyoung Cho, Suwon-si (KR); Jeonggun Lee, Seoul (KR); Beomseok Lee, Hwaseong-si (KR); Jongmyeon Park, Seoul (KR)

Correspondence Address: SUGHRUE MION, PLLC, 2100 PENNSYLVANIA AVENUE, N.W., SUITE 800, WASHINGTON, DC, 20037, US

(73) Assignee: Samsung Electronics Co., Ltd., Suwon-si (KR)

(21) Appl. No.: 12/499,144

(22) Filed: Jul. 8, 2009

(30) Foreign Application Priority Data
Jul. 18, 2008 (KR) 10-2008-0070164

(51) Int. Cl. G01N 35/00; G01N 33/00

(52) U.S. Cl. 436/45; 422/68.1

(57) ABSTRACT

A microfluidic device and a method of transferring a liquid material using the microfluidic device are provided. The microfluidic device includes a chamber storing a sealed liquid material, and a structure that flows air to the chamber, when the structure is rotated to generate centrifugal force, so that the liquid material may be transferred from the chamber.

### CROSS-REFERENCE TO RELATED PATENT APPLICATIONS

This application claims priority from Korean Patent Application No. 10-2008-0070164, filed on Jul. 18, 2008, in the Korean Intellectual Property Office, the disclosure of which is incorporated herein in its entirety by reference.

### BACKGROUND OF THE INVENTION

1. Field of the Invention

Apparatuses and methods consistent with the present invention relate to a centrifugal force-based microfluidic device for performing assays and a method of transferring a liquid material using the same.

2. Description of the Related Art

Microfluidic devices are becoming increasingly important in both research and commercial applications. Microfluidic devices are able to, for example, mix and react reagents in small quantities, thereby minimizing reagent costs. Microfluidic devices also have a relatively small size, thereby saving on laboratory space. Because of their small scale operation, microfluidic devices can be used in various applications. For example, microfluidic devices can be used to quickly and cost effectively perform assays and synthesize products.

Examples of microfluidic devices include rotatory or centrifugal force-based microfluidic devices, such as a rotatory compact disk (CD) based microfluidic device including inlets, outlets, chambers, and vents. In general, a centrifugal force-based microfluidic device includes a substrate capable of rotating around a rotational center. When the centrifugal force-based microfluidic device rotates around the rotational center, centrifugal force can be thought of as being generated therein, and the generated centrifugal force is applied to a liquid material therein, thereby transferring the liquid material from one chamber to another chamber. The centrifugal force-based microfluidic device includes a plurality of chambers disposed in a radial direction away from the rotational center and these chambers may be fluid-communicable with each other through, for example, channels. Also, the substrate is connected to a rotating member. The rotating member can be a motor or a servo motor.

An analyte sample is loaded to and transferred in the centrifugal force-based microfluidic device. Centrifugal force-based microfluidic devices include chambers in which the analyte sample is analyzed, channels, and a liquid material storage chamber containing a liquid material, such as a liquid reagent that is to be used to analyze the analyte sample. Examples of the liquid reagent include a buffer, a diluent, or a reaction reagent. The liquid material storage chamber may optionally include an inlet. When the liquid material storage chamber does not include an inlet, the liquid material is added to the centrifugal force-based microfluidic device only once during manufacture of the device. On the other hand, when the liquid material storage chamber includes an inlet, the liquid material can be repeatedly added to the centrifugal force-based microfluidic device through the inlet. The liquid material is transferred from the liquid material storage chamber to another chamber by centrifugal force generated when the centrifugal force-based microfluidic device rotates. The liquid material storage chamber includes small air vents to aid the transfer of the liquid material by the centrifugal force.

In a related art, the liquid material is un-sealed in a centrifugal force-based microfluidic device. Due to the unsealed nature, the liquid material degrades, and the concentration of the liquid material may change due to evaporation. If the liquid material is sealed, it is not easy to transfer the sealed liquid material in a centrifugal force-based microfluidic device. Accordingly, there is a need to develop a method of stably preserving or efficiently transferring the liquid material in a centrifugal force-based microfluidic device.

### SUMMARY

The present invention provides a centrifugal force-based microfluidic device for stably preserving and/or efficiently transferring a liquid material that is used for a sample assay.

According to an aspect of the present invention, there is provided a microfluidic device including: a substrate to be operated by a centrifugal force; a liquid material storage chamber that is formed in the substrate, contains a liquid material, and is sealed; and a structure which is connected to the liquid material storage chamber and has a valve sealing the structure from the liquid material storage chamber and an outlet through which air is flowable.

The substrate may be operated by centrifugal force. The centrifugal force may be generated by rotating the substrate around a rotational center. The microfluidic device may further include a rotating member for rotating the substrate around the rotation center and/or a control member for controlling the rotating member. The rotating member may be known in the art. In this regard, the rotating member may be a motor or a servo motor. Due to the rotary motion, the centrifugal force is applied to the liquid material away from the rotational center, thereby moving the liquid material in the same direction. Herein, the liquid material may be an analyte sample or a reagent that is used for a sample assay. The liquid material is transferred from a top chamber that is relatively closer to the rotational center to a bottom chamber that is relatively farther from the rotational center through a channel, and the pathway of the liquid material may be controlled by, for example, a fluid transfer control member, such as a valve disposed between chambers or a valve control member. The substrate may be rotated in a clockwise direction or a counter-clockwise direction. The substrate may have various shapes. For example, the substrate may be circular or tetragonal, but the shape of the substrate is not limited thereto.

The liquid material storage chamber is formed in the substrate, contains a liquid material, and is sealed. Herein, the term “sealed” refers to a state in which the liquid material does not substantially contact, for example, external air or gas in the microfluidic device. Such a sealed state may be released by the valve. The liquid material storage chamber may not include a sample inlet. In such a case, the liquid material is loaded once into the liquid material storage chamber and the liquid material storage chamber is sealed. The liquid material may differ according to an analysis reaction occurring in the microfluidic device. For example, the liquid material may be a buffer, a cleansing solution, a reactant, or a detection reagent, but is not limited thereto. Fluid may flow between the liquid material storage chamber and the structure through which air may pass or between the liquid material storage chamber and another chamber, when a valve opens.

The structure may include an outlet through which air may flow when the valve opens, and be connected to the liquid material storage chamber. The structure may be a chamber or a channel. The location and size of the structure may differ according to the location and size of other chambers or channels formed in the substrate. When the structure is a chamber, the chamber may be fluid-communicable with the liquid material storage chamber through, for example, a channel. The structure may be disposed closer to the rotational center than the liquid material storage chamber, or the structure and the liquid material storage chamber may be equidistant from the rotational center but located at different positions, for example adjacent to one another. The structure controls flow of the liquid material contained in the liquid material storage chamber into another chamber or a channel. The controlling operation may be performed with the valve included in the structure. The outlet through which an air flows may have various sizes according to the application purpose of the microfluidic device.

The structure includes the valve and the valve seals the structure. The valve seals or releases the structure and allows air or gas to flow into the liquid material storage chamber containing the liquid material, thereby transferring the liquid material in the liquid material storage chamber. The valve may be formed of a material that is substantially non-transmissible with respect to gas, such as an air. The valve may be any known valve that is used in a microfluidic device. For example, the valve forming material may be a material that changes its state due to electromagnetic irradiation. Such a material may be a phase-transition material or a thermoplastic resin. The phase-transition material may be wax or gel. The valve forming material may include micro heat-dissipating particles that are dispersed in the phase-transition material, and absorb energy of electromagnetic irradiation and dissipate heat. The micro heat-dissipating particles may be selected from a group consisting of metal oxide particles, polymer particles, quantum dots, and magnetic beads. The metal oxide particles may be selected from the group consisting of Al₂O₃, TiO₂, Ta₂O₃, Fe₂O₃, Fe₃O₄ and HfO₂. The micro heat-dissipating particles may have various diameters according to the size of the channel. For example, the micro heat-dissipating particles may have a diameter of 1 nm to 100 μm.

The microfluidic device may further include a valve sealing state-releasing member. The valve sealing state-releasing member may differ according to the type of the valve included in the structure. For example, the valve sealing state-releasing member may use a laser beam or an electron beam to release a sealing state.

The microfluidic device may further include at least one sub-chamber that is connected to the liquid material storage chamber and is configured to contain the liquid material which is contained in the liquid material storage chamber. The sub-chamber may be disposed farther from the rotational center of the substrate than the liquid material storage chamber. When the microfluidic device includes at least two sub-chambers, the shapes of the sub-chambers may be the same as or different from each other. For example, sub-chambers having the same shape may be disposed farther from the rotational center of the substrate than the liquid material storage chamber. The sub-chamber may be fluid-communicable with the liquid material storage chamber through, for example, a channel having a valve. The liquid material contained in the liquid material storage chamber is transferred to the sub-chamber by centrifugal force generated by rotating the substrate by the rotating member while the valve formed between the liquid material storage chamber and the sub-chamber and the valve included in the structure are open by the valve sealing state-releasing member.

The microfluidic device may further include a control member for controlling opening and closing of various valves included in the microfluidic device. Examples of the control member include an electrode plate, an electromagnet, an electronic circuit including various circuit patterns, and an integrated circuit including the electronic circuit. By the control member, valves may allow the liquid material to flow along a desirable pathway.

According to another aspect of the present invention, there is provided a method of transferring a liquid material in a microfluidic device including: a substrate to be operated by centrifugal force; a liquid material storage chamber that is formed in the substrate, contains a liquid material, and is sealed; a structure which has a first valve sealing the structure from the liquid material storage chamber and an outlet through which air is flowable and is connected to the liquid material storage chamber; a sub-chamber that is connected to the liquid material storage chamber and contains the liquid material which has been contained in liquid material storage chamber; and a second valve disposed between the liquid material storage chamber and the sub-chamber, the method including: opening the first and second valves, and transferring the liquid material from the liquid material storage chamber to the sub-chamber by centrifugal force.

A sealing state-releasing member may be used for opening the first and second valves may differ according to the type of the valves. Specifically, the first and second valves may open by, for example, phase transition occurring when exposed to energy, for example, heat energy. In this case, the first and second valves may open by irradiation of a laser beam or an electron beam. The first and second valves may open in any order as long as the valves open when the centrifugal force is applied thereto. For example, the first valve included in the structure may open and then the second valve formed between the liquid material storage chamber and the sub-chamber may open. The centrifugal force may be obtained by rotating the substrate with a rotating member that is coupled to the substrate of the microfluidic device. Due to the rotary motion, centrifugal force is applied to the liquid material contained in the liquid material storage chamber and thus, is transferred from the liquid material storage chamber to the sub-chamber. The substrate may rotate in a clockwise direction or counter-clockwise direction.

The transfer of the liquid material may be controlled with the first and second valves and thus, loss of the liquid material due to evaporation, or degradation of the liquid material in the liquid material storage chamber may be prevented. Also, the transfer path of the liquid material may be appropriately selected.

The substrate of the microfluidic device may be operated by centrifugal force. The centrifugal force may be obtained by rotating the substrate around a rotational center. The microfluidic device may include a rotating member for rotating the substrate around the rotational center and/or a member for controlling the rotating member. The rotating member is known in the art and may be, for example, a motor or a servo motor. Due to the rotary motion, the centrifugal force is applied to the liquid material, such as an analyte sample or a reagent that is used for a sample assay, away from the rotational center, thereby moving the liquid material in the same direction. The liquid material is transferred from a top chamber that is relatively closer to the rotational center to a bottom chamber that is relatively farther from the rotational center through a channel, and the pathway of the liquid material may be controlled by, for example, a fluid transfer control member such as a valve disposed between chambers or a valve control member. The substrate may rotate in a clockwise direction or a counter-clockwise direction. The substrate may have various shapes. For example, the substrate may be circular or tetragonal, but the shape of the substrate is not limited thereto.

The liquid material storage chamber is formed in the substrate, contains a liquid material, and is sealed. Herein, the term “sealed” refers to a state that the liquid material does not substantially contact, for example, external air or gas in the microfluidic device. Such a sealed state may be released by the first and second valves. The liquid material storage chamber may not include a sample inlet. In such a case, the liquid material is loaded once into the liquid material storage chamber and the liquid material storage chamber is sealed. The liquid material may differ depending on an analysis reaction to be performed in the microfluidic device. For example, the liquid material may be a buffer, a cleansing solution, a reactant, or a detection reagent, but is not limited thereto. Fluid may flow between the liquid material storage chamber and the structure through which air may pass when the first valve opens, or fluid may flow between the liquid material storage chamber and the sub-chamber when the second valve opens.

The structure may include an outlet through which air may flow when the valve opens, and be connected to the liquid material storage chamber. The structure may be a chamber or a channel. The location and size of the structure may differ according to the location and size of other chambers or channels formed in the substrate. When the structure is a chamber, the chamber may be fluid-communicable with the liquid material storage chamber through, for example, a channel. The structure may be disposed closer to the rotational center than the liquid material storage chamber, or the structure and the liquid material storage chamber may be equidistant from the rotational center but located at different positions, for example adjacent to one another. The structure controls flow of the liquid material contained in the liquid material storage chamber into another chamber or a channel. The controlling operation may be performed with the valve included in the structure. The outlet through which an air flows may have various sizes according to the application purpose of the microfluidic device.

The structure includes the first valve and the first valve seals the structure. The first valve seals or releases the structure and allows air or gas to flow into the liquid material storage chamber containing the liquid material, thereby transferring the liquid in the liquid material storage chamber. The first valve may be formed of a material that is substantially non-transmissible with respect to gas, such as air. The first valve may be any known valve that is used in a microfluidic device. For example, the valve forming material may be a material that changes its state due to electromagnetic irradiation. Such a material may be a phase-transition material or a thermoplastic resin. The phase-transition material may be wax or gel. The valve forming material may include micro heat-dissipating particles that are dispersed in the phase-transition material, and absorb energy of electromagnetic irradiation and dissipate heat. The micro heat-dissipating particles may be selected from the group consisting of metal oxide particles, polymer particles, quantum dots, and magnetic beads. The metal oxide particles may be selected from the group consisting of A1₂O₃, TiO₂, Ta₂O₃, Fe₂O₃, Fe₃O₄ and HfO₂. The micro heat-dissipating particles may have various diameters depending on the size of the channel. For example, the micro heat-dissipating particles may have a diameter of 1 nm to 100 μm.

The microfluidic device may further include a valve sealing state-releasing member. The valve sealing state-releasing member may differ according to type of the first valve included in the structure. For example, the valve sealing state-releasing member may use a laser beam or an electron beam to release a sealing state.

The microfluidic device may further include at least one sub-chamber that is connected to the liquid material storage chamber and is configured to contain the liquid material which is contained in liquid material storage chamber. The sub-chamber may be disposed farther from the rotational center of the substrate than the liquid material storage chamber. When the microfluidic device includes at least two sub-chambers, the shapes of the sub-chambers may be the same as or different from each other. For example, sub-chambers having the same shape may be disposed farther from the rotational center of the substrate than the liquid material storage chamber. The sub-chamber may be fluid-communicable with the liquid material storage chamber through, for example, a channel having the second valve. The liquid material contained in the liquid material storage chamber is transferred to the sub-chamber by centrifugal force generated by rotating the substrate by the rotating member while the second valve formed between the liquid material storage chamber and the sub-chamber and the first valve included in the structure are open by the valve sealing state-releasing member.

The microfluidic device may further include a control member for controlling opening and closing of various valves included in the microfluidic device. Examples of the control member include an electrode plate, an electromagnet, an electronic circuit including various circuit patterns, and an integrated circuit including the electronic circuit. By way of the control member, valves can allow the liquid material to flow along a desirable pathway.

### BRIEF DESCRIPTION OF THE DRAWINGS

The above and other aspects of the present invention will become more apparent by describing in detail exemplary embodiments thereof with reference to the attached drawings, in which:

FIG. 1 is a schematic view of a microfluidic device including a structure that includes an air vent, according to an exemplary embodiment of the present invention;

FIG. 2 illustrates how a liquid material is transferred in the microfluidic device of FIG. 1, according to an exemplary embodiment of the present invention;

FIG. 3 is a schematic view of a microfluidic device according to another exemplary embodiment of the present invention, wherein a structure including an air vent is disposed next to a liquid material storage;

FIG. 4 is a schematic view of a microfluidic device according to another exemplary embodiment of the present invention, including at least two sub-chambers; and

FIG. 5 is a schematic view of a microfluidic device according to another exemplary embodiment of the present invention, including a substrate being operated by centrifugal force.

### DETAILED DESCRIPTION

The present invention will now be described more fully with reference to the accompanying drawings, in which exemplary embodiments of the invention are shown.

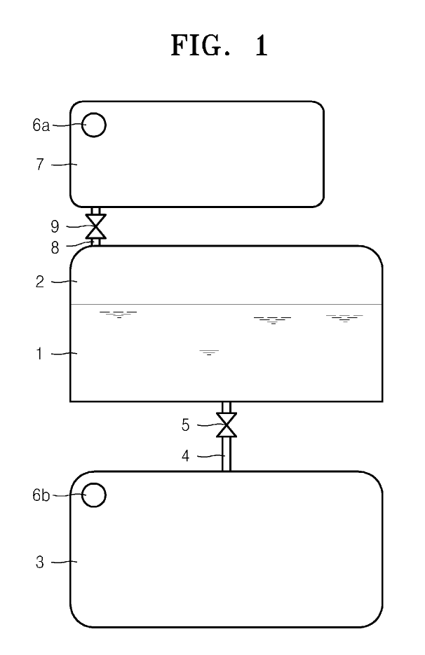

FIG. 1 is a schematic view of a microfluidic device including a structure 7 that includes an air vent 6a, according to an exemplary embodiment of the present invention.

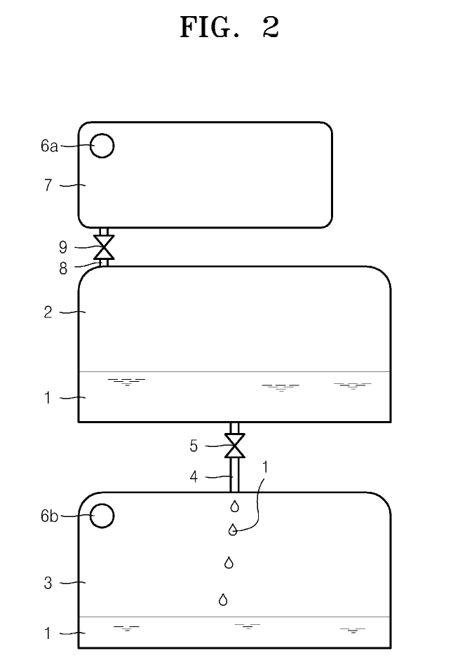

Referring to FIG. 1, the microfluidic device according to the exemplary embodiment includes the structure 7, a liquid material storage chamber 2, a sub-chamber 3, and a valve 4 formed between the liquid material storage chamber 2 and the sub-chamber 3. The structure 7 is formed in a substrate (not shown) to be operated by centrifugal force, includes a valve 9 sealing the structure 7 and the air vent 6a through which an air flows, and is connected to the liquid material storage chamber 2. The liquid material storage chamber 2 contains a liquid material 1 and is sealed. The sub-chamber 3 is connected to the liquid material storage chamber 2 and is to contain the liquid material 1 that has been contained in the liquid material storage chamber 2. The structure 7 may be fluid-communicable with the liquid material storage chamber 2 through a channel 8. It should be noted the top part of FIG. 1 is closer to a rotational center of the microfluidic device than the bottom part. That is, when the device is rotated centrifugal force will be generated that acts in a direction from the top part of FIG. 1 to the bottom part of FIG. 1. This orientation is maintained in FIGS. 2-4. In FIG. 1, the structure 7 is a chamber including the channel 8 that includes the valve 9. However, in some exemplary embodiments, the structure 7 can be a channel including a valve. The sub-chamber 3 may be fluid-communicable with the liquid material storage chamber 2 through the channel 4 including a valve 5. The sub-chamber 3 may include an air vent 6b. Since, as illustrated in FIG. 1, the structure 7 including the air vent 6a is fluid-communicable with the liquid material storage chamber 2 through the channel 8 including the valve 9, the liquid material 1 contained in the liquid material storage chamber 2 can be transferred by opening and closing the valve 9 and the liquid material 1 can be preserved without loss. Although not illustrated, the microfluidic device may further include a rotating member for rotating the substrate. The rotation member can be a motor or a servo motor.

The valves 5 and 9 may be formed of a material that changes its state by electromagnetic irradiation. The valve forming material may be a material that changes its state by energy. Such a material can be a phase-transition material or a thermoplastic resin. The phase-transition material may be wax or gel. The valve forming material may include micro heat-dissipating particles that are dispersed in the phase-transition material, and absorb energy of electromagnetic irradiation and dissipate heat. The micro heat-dissipating particles may be selected from a group consisting of metal oxide particles, polymer particles, quantum dots, and magnetic beads. The metal oxide particles may be selected from the group consisting of A1₂O₃, TiO₂, Ta₂O₃, Fe₂O₃, Fe₃O₄ and HfO₂. The micro heat-dissipating particles may have various diameters according to the sizes of the channels 4 and 8. For example, the micro heat-dissipating particles may have a diameter of 1 nm to 100 μm.

FIG. 2 illustrates how the liquid material 1 is transferred in the microfluidic device of FIG. 1, according to an exemplary embodiment of the present invention.

Referring to FIG. 2, the liquid material 1 is transferred from the liquid material storage chamber 2 to the sub-chamber 3 by applying centrifugal force to the substrate of the microfluidic device while the valves 5 and 9 are open. The opening order of the valves 5 and 9 is not limited. In this exemplary embodiment, the structure 7 controls flow of the liquid material 1 from the liquid material storage chamber 2 through the valve 5 using the centrifugal force and air that flows through the valve 9.

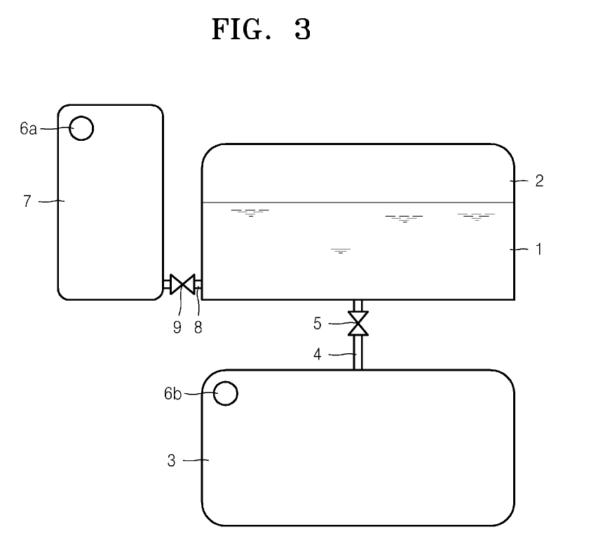

FIG. 3 is a schematic view of a microfluidic device according to another embodiment of the present invention, in which a structure 7 including an air vent 6a is disposed next to a liquid material storage chamber 2.

Referring to FIG. 3, the structure 7 including the air outlet 6a is disposed next to the liquid material storage chamber 2, and is fluid-communicable with the liquid material storage chamber 2 through a channel 8 including a valve 9. Since the structure 7 is located next to the liquid material storage chamber 2, the space of the substrate of the microfluidic device can be effectively used and the weight of the microfluidic device can be reduced.

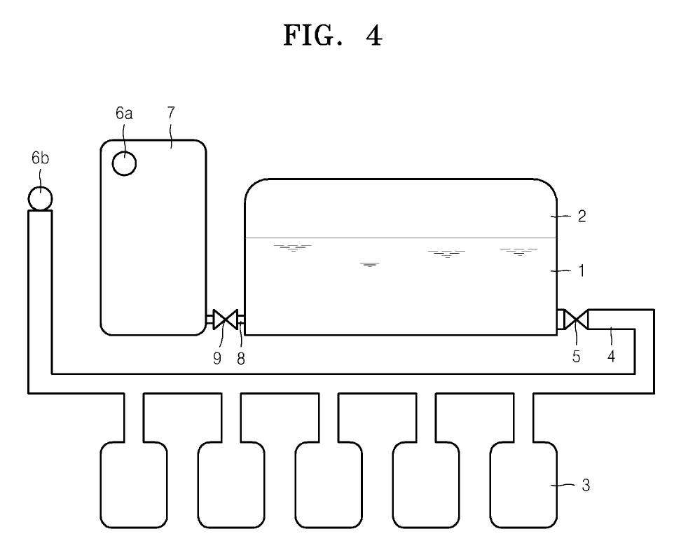

FIG. 4 is a schematic view of a microfluidic device according to another embodiment of the present invention, including at least two sub-chambers 3.

Referring to FIG. 4, the sub-chambers 3 are connected to a liquid material storage chamber 2 through a channel 4 including a valve 5, and are disposed further from the rotational center of the device than the liquid material storage chamber 2.

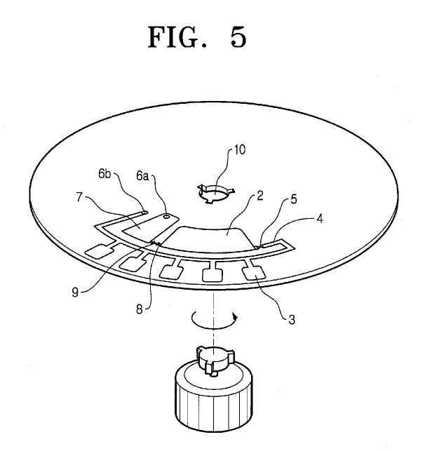

FIG. 5 is a schematic view of a microfluidic device according to another embodiment of the present invention, including a substrate to be operated by centrifugal force.

Referring to FIG. 5, the microfluidic device includes: a liquid material storage chamber 2; a structure 7 that is fluid-communicable with the liquid material storage chamber 2 through a channel 8 including a valve 9, and includes an air vent 6a; and a plurality of sub-chambers 3 that are connected to the liquid material storage chamber 2 through a channel 4 including a valve 5, and includes an air vent 6b, wherein the liquid material storage chamber 2, the structure 7, and the sub-chambers 3 are formed in a disc-shaped substrate. A rotating member of the substrate is coupled to the substrate at a rotating member binding site 10 of the substrate. The rotating member may rotate the substrate, for example, in a counter-clockwise direction, thereby generating rotary motion. The generated rotary motion affects a liquid material contained in the liquid material storage chamber 2 to transfer the liquid material into each sub-chamber 3. Each sub-chamber 3 may include a valve (now shown). Due to inclusion of the air vent 6a and the channel 8 including the valve 9, the structure 7 is fluid-communicable with the liquid material storage chamber 2 through the channel 8 including the valve 9, and thus the liquid can be stably preserved and efficiently transferred in the liquid material storage chamber.

As described above, a liquid material can be efficiently transferred in the microfluidic devices according to the exemplary embodiments of the present invention and also, the same effect can be obtained by using the methods according to the embodiments of the present invention.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A microfluidic device comprising:
a substrate that generates centrifugal force when the substrate is rotated;
a liquid material storage chamber that is formed in the substrate, contains a liquid material, and is sealed; and
a structure connected to the liquid material storage chamber, the structure comprising:
a first fluid transfer control member that seals the structure from the liquid material storage chamber; and
an outlet through which air is flowable and which connects the structure and the liquid material storage chamber through the first fluid transfer control member,
wherein the structure controls flow of the liquid material from the liquid material storage chamber through the first fluid transfer control member using the centrifugal force and the air.

2. The microfluidic device of claim 1, wherein the structure is a channel or a chamber.

3. The microfluidic device of claim 1, further comprising at least one sub-chamber that is connected to the liquid material storage chamber and is configured to receive and contain the liquid material which is contained in the liquid material storage chamber.

4. The microfluidic device of claim 3, wherein the at least one sub-chamber are connected to each other and equidistant from a rotational center of the substrate.

5. The microfluidic device of claim 3, further comprising a second fluid transfer control member disposed between the liquid material storage chamber and the sub-chamber.

6. The microfluidic device of claim 5, at least one of the first and second fluid transfer control members is a valve.

7. The microfluidic device of claim 6, wherein the valve is formed of a material that changes a state of the material by electromagnetic irradiation on the material.

8. The microfluidic device of claim 1, wherein the structure is disposed upstream of the liquid material storage chamber with respect to a rotational center of the substrate, or the structure is disposed next to the liquid material storage chamber with respect to a rotational center of the substrate.

9. The microfluidic device of claim 1, wherein the first fluid transfer control member is a valve formed of a material that changes a state of the material by electromagnetic irradiation on the material.

10. The microfluidic device of claim 9, wherein the material is selected from a phase-transition material and a thermoplastic resin.

11. The microfluidic device of claim 9, wherein the material comprises a phase-transition material, and micro heat-dissipating particles that are dispersed in the phase-transition material, absorb energy of electromagnetic irradiation and dissipate heat.

12. The microfluidic device of claim 1, wherein the liquid material comprises an analyte sample, a reagent, reactant, a buffer solution or a cleansing solution that is used for a sample assay.

13. A device for controlling flow of a liquid material, the device comprising:
a structure that receives centrifugal force and comprises a first control member;
a first chamber that contains a liquid material sealed from an outside and comprises a second control member;
a second chamber that is connected to the first chamber,
wherein the centrifugal force flows air into the first chamber through the first control member so that the liquid material can flow to the second chamber through the second control member.

14. A method of transferring a liquid material in a microfluidic device comprising a structure, a substrate in which a liquid material storage chamber containing a liquid material is formed, and a sub-chamber, the method comprising:
controlling a first fluid transfer control member, which is disposed between the structure and the liquid material storage chamber and seals the structure from the liquid material storage chamber, to release the structure from the sealing;
controlling a second fluid transfer control member, which is disposed between the liquid material storage chamber and the sub-chamber, to connect the liquid material storage chamber and the sub-chamber;
generating centrifugal force by rotating the substrate with respect to a rotational center of the substrate and flowing air into the structure so that the liquid material is transferred from the liquid material storage chamber to the sub-chamber using the centrifugal force and the air flow.

15. The method of claim 14, wherein the structure is a channel or a chamber.

16. The method of claim 14, wherein the structure is disposed closer to the rotational center than the liquid material storage chamber, or the structure is disposed at a same distance from the rotational center as the liquid material storage chamber.

17. The method of claim 14, wherein at least one of the first and second fluid transfer control members is a valve.

18. The method of claim 17, wherein the valve is formed of a material that changes a state of the material by electromagnetic irradiation on the material.

19. The method of claim 18, wherein the material is selected from a phase-transition material and a thermoplastic resin.

20. The method of claim 18, wherein the material comprises a phase-transition material, and micro heat-dissipating particles that are dispersed in the phase-transition material, absorb energy of electromagnetic irradiation and dissipate heat.
